(19) United States
(12) Patent Application Publication
(10) Pub. No.: US 2023/0414907 A1
(43) Pub. Date: Dec. 28, 2023

(54) CATHETER AND METHOD OF ACTUATING BALLOON

(71) Applicant: Japan Lifeline Co., Ltd., Tokyo (JP)

(72) Inventors: Yasuhiro OKAWA, Tokyo (JP); Yusuke HAYAMI, Tokyo (JP)

(21) Appl. No.: 18/341,049

(22) Filed: Jun. 26, 2023

(30) Foreign Application Priority Data
Jun. 28, 2022 (JP) 2022-103687
Apr. 20, 2023 (JP) 2023-069452

(51) Int. Cl. A61M 25/10

(52) U.S. Cl. CPC A61M 25/1029; A61M 25/1002; A61M 25/1034; A61M 2025/1031

(57) ABSTRACT

A catheter according to an aspect of the present disclosure includes a shaft, an inflatable balloon attached to a distal end side of the shaft, and a covering member configured to cover an outer periphery of the balloon and to be elastically deformable in conformity with deflation of the balloon. When the balloon is inflated and then deflated, the covering member is deflated to a state before inflation by elastic deformation, in conformity with the deflation of the balloon. Thus, the maximum diameter of the balloon in the deflated state can be reduced.

### CROSS REFERENCE TO RELATED APPLICATIONS

This application claims the benefit of priority to Japanese Patent Application Number 2022-103687 filed on Jun. 28, 2022. The entire contents of the above-identified application are hereby incorporated by reference.

### TECHNICAL FIELD

The present disclosure relates to a catheter, and more specifically to a balloon catheter including an inflatable balloon.

### BACKGROUND

A catheter is a medical tube that is inserted into a body for inspection or treatment. More particularly, a catheter including a balloon that can be expanded in the body is referred to as a balloon catheter and is used for dilating a dilatation target part or a constricted part (hereinafter, referred to as “constricted part or the like”) in: a tubular organ in the body such as a blood vessel, trachea, gastrointestinal tract, common bile duct, and pancreatic ductus; a connection part (inlet and outlet) between these; a hole formed in the body for inspection or treatment (e.g., a hole that punctures the common bile duct from the stomach or the duodenal bulb); and the like.

Conventionally, a balloon catheter having the structure described in JP 2002-028243 A is known.

### SUMMARY

After the balloon dilates the constricted part or the like, the balloon is deflated and pulled out with the shaft from the dilated constricted part or the like. In this case, when the maximum diameter of the balloon in the deflated state is large, it is difficult to remove the balloon.

The present disclosure has been made in view of such circumstances, and an object thereof is to provide a catheter with which a maximum diameter of a balloon in a deflated state can be reduced.

A catheter according to an aspect of the present disclosure for solving the problem described above includes: a shaft; a balloon that is inflatable and attached to a distal end side of the shaft; and a covering member configured to cover an outer periphery of the balloon and to be elastically deformable in conformity with deflation of the balloon.

Another aspect of the present disclosure is a method of actuating a balloon. The method includes inflating the balloon together with a covering member configured to cover an outer periphery of the balloon by supplying a fluid to an inside of the balloon, and deflating the balloon together with the covering member by discharging the fluid to an outside of the balloon.

According to the catheter of the present disclosure, it is possible to reduce the maximum diameter of the balloon in the deflated state.

### BRIEF DESCRIPTION OF DRAWINGS

FIGS. 1A, 1B, and 1C schematically illustrate an appearance of a balloon catheter.

FIG. 2 is a cross-sectional view taken along line A-A in FIG. 1B.

FIGS. 3A, 3B, and 3C are cross-sectional views taken along lines B-B, C-C, and D-D in FIGS. 1A to 1C.

FIGS. 4A, 4B, and 4C schematically illustrate a method of manufacturing a balloon.

### DESCRIPTION OF EMBODIMENTS

Hereinafter, embodiments of the present disclosure will be described in detail with reference to the drawings. In the description or the drawings, the same or equivalent constituent elements, members, and processing operations are denoted by the same reference signs, and redundant descriptions are omitted. The scales and shapes of the illustrated parts are set for convenience to facilitate the explanation and should not be construed as limiting unless otherwise specified. The embodiments are illustrative and do not limit the scope of the present disclosure in any way. Not all features or combinations of said features described in the embodiments are essential to the present disclosure.

A catheter of the present disclosure can be used to dilate a constricted part or the like at any location.

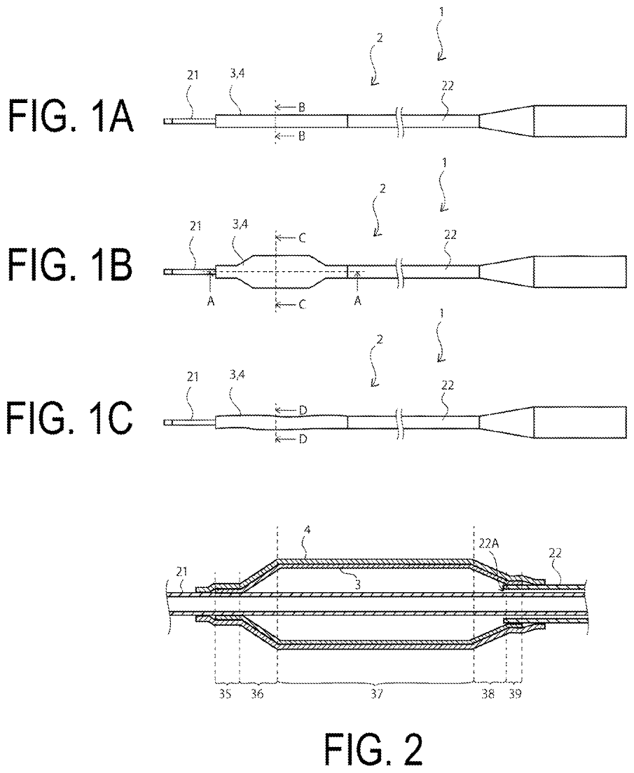

FIGS. 1A to 1C illustrate an appearance of a balloon catheter 1 of the present disclosure. The balloon catheter 1 includes a long and tubular flexible shaft 2, and a balloon 3 that can be inflated and deflated. The balloon 3 is attached to a distal end side of the shaft 2 (distal side with respect to half of the entire length of the shaft 2, i.e., distal side with respect to a center of the shaft 2 in a longitudinal direction). As to be described in detail later, the balloon 3 assumes an unused state, an inflated state, and a deflated state. The balloon 3 in FIG. 1A is in an unused state, the balloon 3 in FIG. 1B is in an inflated state, and the balloon 3 in FIG. 1C is in a deflated state.

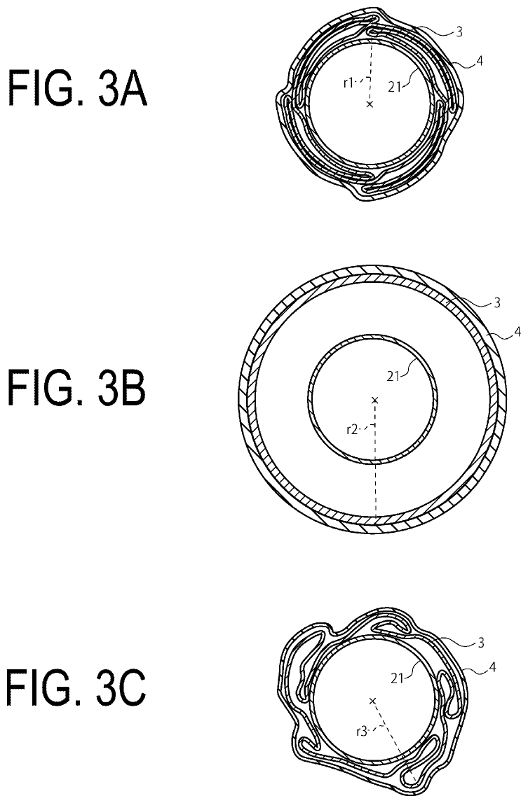

FIG. 2 is a cross-sectional view taken along line A-A in FIG. 1B. FIG. 3A is a cross-sectional view taken along line B-B in FIG. 1A, FIG. 3B is a cross-sectional view taken along line C-C in FIG. 1B, and FIG. 3C is a cross-sectional view taken along line D-D in FIG. 1C. As illustrated in FIGS. 2A to 3C, the shaft 2 includes an inner shaft 21 and an outer shaft 22. The inner shaft 21 is inserted into (the lumen of) the outer shaft 22. A distal end portion of the balloon 3 is fixed to an outer periphery of the inner shaft 21, and a proximal end portion of the balloon 3 is fixed to an outer periphery of the outer shaft 22. One or more balloon expansion lumens 22A are formed between the outer periphery of the inner shaft 21 and an inner periphery of the outer shaft 22. The balloon expansion lumen 22A communicates with the inside of the balloon 3.

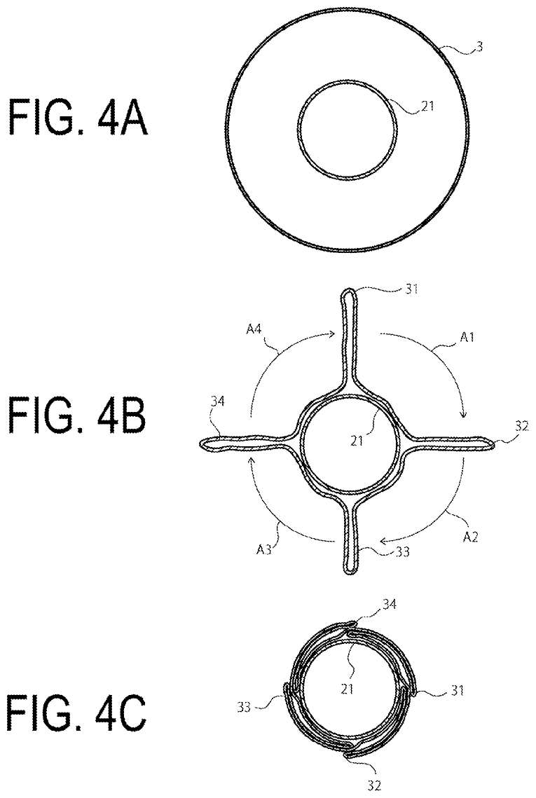

FIGS. 4A to 4C schematically illustrate a method of manufacturing or a method of forming the balloon 3. More specifically, FIGS. 4A to 4C illustrate a series of operations or processes performed when folding the balloon 3 in the method of manufacturing the balloon 3. Before the balloon 3 is folded, the balloon 3 is in an inflated state due to a fluid (e.g., air) enclosed inside the balloon 3 (refer to FIG. 4A). When the fluid is drawn out from the inside of the balloon 3 in the state of FIG. 4A, the balloon 3 starts to deflate. In this case, a plurality of (four, in the illustrated example) protruding portions 31 to 34 or projecting portions are formed at substantially equal intervals (at intervals of about 90°, in the shown example) in a circumferential direction of the balloon 3 by using a predetermined guide (not illustrated). Each of the protruding portions 31 to 34 has a shape protruding radially from a recessed portion between corresponding protruding portions 31 to 34 about the inner shaft 21 (refer to FIG. 4B). As illustrated in FIG. 4C, the guide is moved such that the protruding portions 31 to 34 are folded along the circumferential direction of the inner shaft 21 (refer to arrows A1, A2, A3, and A4 in FIG. 4B), thereby deflating or compactly folding the entire balloon 3. Thereafter, as illustrated in FIG. 3A, a covering member 4 is placed on an outer periphery side of the balloon 3 in the entirely deflated (compactly folded) state, thereby completing the balloon 3 in the unused state.

### Balloon 3

As illustrated in FIGS. 1A, 3A, and 4C, the balloon 3 in the unused state has a shape in which the entire balloon 3 is deflated in a manner by which the protruding portions 31 to 34 are folded about the inner shaft 21. The unused state refers to a state immediately after the balloon catheter 1 is manufactured (e.g., immediately after the balloon 3 and the covering member 4 are combined), that is, a state in which the balloon 3 has not yet been inflated after manufacture.

As illustrated in FIGS. 1B, 2, and 3B, the balloon 3 in the inflated state has such a shape that a cross section (FIG. 3B) perpendicular to the longitudinal direction (left-right direction in FIG. 2) of the shaft 2 or the balloon 3 is circular. When a fluid is supplied to the inside of the balloon 3 in the unused state illustrated in FIG. 3A, the folded protruding portions 31 to 34 rise in the radial direction and expand in the circumferential direction, whereby the balloon 3 is inflated. That is, a diameter of the balloon 3 in the inflated state (e.g., the maximum diameter r2 in FIG. 3B) is larger than a diameter of the balloon 3 in the unused state (e.g., the maximum diameter r1 in FIG. 3A).

As illustrated in FIG. 2, the balloon 3 in the inflated state includes, from a distal end toward a proximal end, a distal end-side neck portion 35, a distal end-side tapered portion 36, an intermediate portion 37, a proximal end-side tapered portion 38, and a proximal end-side neck portion 39. The distal end-side tapered portion 36 is formed to have a tapered shape with a diameter increasing toward the intermediate portion 37 from the distal end-side neck portion 35, whose inner periphery has substantially the same diameter as that of the outer periphery of the inner shaft 21. The proximal end-side tapered portion 38 is formed to have a tapered shape with a diameter increasing toward the intermediate portion 37 from the proximal end-side neck portion 39, whose inner periphery has substantially the same diameter as that of the outer periphery of the outer shaft 22.

In the illustrated example where an expansion diameter of a proximal end of the distal end-side tapered portion 36 and an expansion diameter of a distal end of the proximal end-side tapered portion 38 are substantially equal, the intermediate portion 37 coupling these portions in the longitudinal direction serves as a straight tube portion having a substantially uniform expansion diameter. Note that, in the illustrated example, a length of the distal end-side tapered portion 36 and a length of the proximal end-side tapered portion 38 are substantially equal in the longitudinal direction of the shaft 2, but may be significantly different from each other. The inflatable or expandable balloon 3 is fixed to the shaft 2 by fixing an inner peripheral surface of the distal end-side neck portion 35 to an outer peripheral surface of the inner shaft 21 and fixing an inner peripheral surface of the proximal end-side neck portion 39 to an outer peripheral surface of the outer shaft 22.

As illustrated in FIGS. 1C and 3C, the balloon 3 in the deflated state has a diameter smaller than that in the inflated state in a cross section perpendicular to the longitudinal direction of the shaft 2. For example, a maximum diameter (r3 in FIG. 3C) of the balloon 3 in the deflated state is smaller than the maximum diameter (r2 in FIG. 3B) of the balloon 3 in the inflated state. The deflated state refers to a state in which the fluid inside the balloon 3 is removed from the inflated state (i.e., a state in which the balloon 3 that was inflated once has been deflated by removing the fluid).

When the balloon 3 is changed from the inflated state to the deflated state, the protruding portions 31 to 34 may not be neatly (i.e., regularly) folded, as compared with the unused state. Therefore, unevenness of the balloon outer peripheral surface due to the protruding portions 31 to 34 may be more prominent or more irregular than unevenness in the unused state. That is, the maximum diameter (e.g., r3 in FIG. 3C) of the balloon 3 in the deflated state may be larger than the maximum diameter (e.g., r1 in FIG. 3A) of the balloon 3 in the unused state. This is because larger gaps or more gaps are formed between the protruding portions 31 to 34, as compared with the unused state in which the balloon 3 is neatly folded. However, as to be described later, since the covering member 4 provided on the outer periphery side of the balloon 3 suppresses excessive protrusion of the protruding portions 31 to 34 (i.e., reduces the maximum diameter r3 of the balloon 3), smooth removal of the balloon 3 is not impeded.

The maximum diameter r1 of the balloon 3 in the unused state is about 0.5 to 2.0 mm. The maximum diameter r2 of the balloon 3 (intermediate portion 37) in the inflated state is about 2.0 to 9.0 mm. The maximum diameter r3 of the balloon 3 in the deflated state is about 0.5 to 3.0 mm. Here, the maximum diameters r1, r2, and r3 are distances (radii) from the center of the inner shaft 21 (or the center of the shaft 2) to the most distant position on the outer surface or outer peripheral surface of the balloon 3. The balloon 3 is made of any resin material such as nylon, nylon-based elastomer, polyurethane, or polyethylene terephthalate.

### Covering Member 4

The covering member 4 is elastically deformable and has a long and tubular shape. The covering member 4 covers the outer periphery of the balloon 3. Here, “the covering member 4 covers the outer periphery of the balloon 3” means that the covering member 4 is arranged covering the outer periphery of at least a main portion (portion to be inflated) of the balloon 3. Note that, in the unused state of the balloon 3 illustrated in FIG. 3A and in the deflated state of the balloon 3 illustrated in FIG. 3C, since the balloon 3 is folded, an inner peripheral surface of the covering member 4 is in contact with or close to a part of the outer peripheral surface of the main portion of the balloon 3. In addition, in the inflated state of the balloon 3 illustrated in FIG. 3B, since the balloon 3 is inflated, the inner peripheral surface of the covering member 4 is in contact with or close to substantially the entire outer peripheral surface of the main portion of the balloon 3. Note that another member may be interposed between the covering member 4 on the outer periphery side and the balloon 3 on the inner periphery side. Even in this case, the covering member 4 covers the outer periphery of the balloon 3, but at least a part of the inner peripheral surface of the covering member 4 is in contact with an outer peripheral surface of the other member, instead of being in contact with the outer peripheral surface of the balloon 3.

In the example illustrated in FIG. 2, the main portion of the balloon 3 includes the intermediate portion 37. The main portion may further include the distal end-side tapered portion 36 and/or the proximal end-side tapered portion 38. In addition, only the vicinities of both ends (end portions) of the covering member 4 are fixed to the outer peripheral surface of the shaft 2 or the balloon 3 (portions other than the main portion (e.g., the distal end-side neck portion 35 and the proximal end-side neck portion 39)). The distal end portion of the covering member 4 is fixed by a short fixing tube made of thermoplastic elastomer or the like, in a state of covering the outer periphery of the inner shaft 21 extending toward the distal end side of the balloon 3. Further, the proximal end portion of the covering member 4 is fixed by a short fixing tube made of thermoplastic elastomer or the like, in a state of covering the outer periphery of the outer shaft 22 extending toward the proximal end side of the balloon 3. A length of the covering member 4 (length along the shaft 2) is preferably equal to or longer than a length of the balloon 3 in a non-inflated state in which no tensile force acts on the covering member 4, or the like. Note that the distal end portion of the covering member 4 may be fixed to the outer peripheral surface of the distal end-side neck portion 35 (fixed to the outer peripheral surface of the inner shaft 21) at the distal end portion of the balloon 3, and the proximal end portion of the covering member 4 may be fixed to the outer peripheral surface of the proximal end-side neck portion 39 (fixed to the outer peripheral surface of the outer shaft 22) at the proximal end portion of the balloon 3.

In the unused state of the balloon 3, the covering member 4 has a shape conforming to the outer periphery of the balloon 3, as illustrated in FIGS. 1A and 3A. In this case, the covering member 4 presses the balloon 3 in a direction (toward the inner periphery side) in which the diameter of the balloon 3 is reduced (i.e., the maximum diameter r1 is suppressed) by elastic force.

In the inflated state of the balloon 3, the covering member 4 has a shape conforming to the outer periphery of the inflated balloon 3, as illustrated in FIGS. 1B, 2, and 3B. The covering member 4 is inflated with the inflation of the balloon 3. The material or properties (such as Young's modulus) of the covering member 4 are preferably selected so as not to interfere with inflation of the balloon 3. The expression “does not interfere with inflation of the balloon 3” means that the balloon 3 is allowed to inflate, and does not mean that the balloon 3 is not resistant to expansion. Specifically, the covering member 4 is made of any elastic material, such as polyurethane. The elastic modulus of the covering member 4 is smaller than the elastic modulus of the balloon 3. Since the elastic modulus (also known as Young's modulus) represents resistance to deformation, the balloon 3 can be regarded as less deformable than the covering member 4. That is, when the balloon 3 on an inner side is inflated and deflated, the covering member 4 that is more deformable than the balloon 3 deforms in conformity with the inflation and deflation of the balloon. In this way, the balloon 3 that is less deformable leads the inflation operation and deflation operation of the balloon catheter 1, and the covering member 4 that is more deformable follows the inflation operation and deflation operation. As to be described later, the covering member 4 functions to suppress the maximum diameter of the balloon 3 particularly in the unused state and the deflated state.

In the deflated state of the balloon 3, the covering member 4 has a shape conforming to the outer periphery of the balloon 3, as illustrated in FIGS. 1C and 3C. The covering member 4 is deflated with the deflation of the balloon 3. In this case, the covering member 4 presses the balloon 3 in a direction (toward the inner periphery side) in which the diameter of the balloon 3 is reduced (i.e., the maximum diameter r3 is suppressed) by elastic force. As a result, for example, gaps between the protruding portions 31 to 34 and the amount of radial protrusion (i.e., the maximum diameter r3) of the protruding portions 31 to 34 are reduced, as compared with a case where the covering member 4 is not provided.

A lubricant of any material such as silicone may be added or applied between the inner periphery of the covering member 4 and the outer periphery of the balloon 3 (in particular, at the main portion to be inflated such as the intermediate portion 37). Thereby, the balloon 3 and the covering member 4 can be inflated or deflated at the same time without interfering with each other due to friction or the like.

It is preferable that as little air as possible be present between the balloon 3 and the covering member 4. However, a minute air layer or gap may be formed depending on the use (i.e., the application site of the balloon catheter 1).

Hereinafter, the action or operation of the balloon catheter 1 will be described. As illustrated in FIG. 3A, before the balloon catheter 1 is used, the balloon 3 is in a state (unused state) of being deflated and wound on the inner shaft 21. In addition, the elastically deformable covering member 4 covers the outer periphery of the balloon 3 in the unused state, and the maximum diameter r1 is small. As illustrated in FIGS. 2 and 3B, the balloon 3 in the unused state is inflated as a fluid is supplied to the inside of the balloon 3 (via the balloon expansion lumen 22A). The covering member 4 also inflates with the inflation of the balloon 3. As illustrated in FIG. 3C, the balloon 3 in the inflated state is deflated as the fluid supplied to the inside is discharged to the outside (via the balloon expansion lumen 22A). The covering member 4 also deflates with the deflation of the balloon 3. In this case, since the covering member 4 is deflated by elastic deformation and presses the balloon 3 in the deflated state from the outer diameter side or outer periphery side to the inner diameter side or inner periphery side toward the center of the inner shaft 21, the maximum diameter r3 of the balloon 3 is reduced. This makes it easy to remove the balloon catheter 1 or the balloon 3.

Even in the inflated state illustrated in FIG. 3B, the covering member 4 presses the balloon 3 from the outer diameter side or outer periphery side to the inner periphery side toward the center of the inner shaft 21. Thus, the covering member 4 can also be interpreted as a suppressing member that suppresses the balloon 3 from being excessively inflated or an adjusting member that appropriately adjusts the maximum diameter r2 of the balloon 3 in the inflated state.

In order to achieve the inflated states of the balloon 3 and the covering member 4 illustrated in FIGS. 1B, 2, and 3B, the elastic modulus of the covering member 4 is made smaller than the elastic modulus of the balloon 3.

### Method of Measuring Elastic Modulus

The elastic modulus was calculated by performing a tensile test on the balloon 3 and the covering member 4. Both end portions of the balloon 3 or the covering member 4 were fixed by gripping tools, and a pulling operation was carried out so that the gripping tools separated at a speed of 200 mm/min. In the test results, the elastic modulus (N/mm²) can be calculated by dividing the pulling force (N) by the square of the separation distance (mm) of the gripping tool. The elastic modulus of the balloon 3 was 250 to 300 N/mm² and the elastic modulus of the covering member 4 was 1 to 5 N/mm².

As illustrated in FIGS. 3A and 3C, the covering member 4 covers the outer periphery of the deflated balloon 3. That is, irregularities (wrinkles) are less likely to occur on the outer surface of the covering member 4. This facilitates insertion and removal of the balloon catheter 1.

The present disclosure has been described above based on the embodiments. It should be understood by those skilled in the art that the embodiments are examples, that various modifications are possible in the combination of components and processing operations, and that such modifications are also within the scope of the present disclosure.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. A catheter comprising:
a shaft;
a balloon that is inflatable and attached to a distal end side of the shaft; and
a covering member configured to cover an outer periphery of the balloon and to be elastically deformable in conformity with deflation of the balloon.

2. The catheter according to claim 1, wherein an elastic modulus of the covering member is smaller than an elastic modulus of the balloon.

3. The catheter according to claim 1, wherein only both end portions of the covering member are fixed to a portion other than a main portion of the balloon or to the shaft.

4. The catheter according to claim 1, wherein the covering member covers the outer periphery of the balloon in an unused state.

5. A method of actuating a balloon, the method comprising:
inflating the balloon together with a covering member configured to cover an outer periphery of the balloon by supplying a fluid to an inside of the balloon; and
deflating the balloon together with the covering member by discharging the fluid to an outside of the balloon.
